(19) United States
(12) Patent Application Publication
(10) Pub. No.: US 2012/0237600 A1
(43) Pub. Date: Sep. 20, 2012

(54) MULTILAYER PHARMACEUTICAL COMPOSITION THAT CAN BE DISPERSED IN WATER AND WHICH CONTAINS A COMBINATION OF ANTIMALARIAL AGENTS

(75) Inventors: Jonathan CHAN-SEW, Montpellier (FR); Frantz ELBAZ, Montpellier (FR)

(73) Assignee: SANOFI, Paris (FR)

(21) Appl. No.: 13/459,706

(22) Filed: Apr. 30, 2012

Related U.S. Application Data
(63) Continuation of application No. PCT/FR2010/052369, filed on Nov. 4, 2010.

(30) Foreign Application Priority Data
Nov. 5, 2009 (FR) 0905299

(51) Int. Cl. A61K 9/20; A61P 33/06; A61K 31/555

(52) U.S. Cl. 424/465; 514/187

(57) ABSTRACT

The present invention relates to a multilayer pharmaceutical composition that can be dispersed in water, containing one antimalarial agent in combination with at least one other antimalarial agent. The present invention also relates to a method for producing such a pharmaceutical composition.

The present invention relates to a water-dispersible multilayer pharmaceutical composition comprising an antimalarial agent in combination with at least one other antimalarial agent. The present invention also comprises a method for producing such a pharmaceutical composition.

Each year, malaria causes 400 to 900 million cases of fever, and between one and three million deaths, i.e., on average, one death every 30 seconds. The vast majority of victims are children under the age of 5 and pregnant women.

Despite the efforts undertaken to reduce transmission of the disease and to improve its treatment, there has been little change since the beginning of the 1990s. It is also estimated that, if the prevalence of malaria continues at its current rate, the mortality rate could double in the next twenty years.

The strong upsurge in the disease observed over the last few years is due to several factors, including:
the vectors, namely anopheles mosquitoes, which become resistant to conventional and inexpensive insecticides, such as DDT (abbreviation for 1,1,1-trichloro-2,2-bis(p-chlorophenyl)ethane);
the increase in the population in the areas at risk and, mainly,
the resistance of many strains of Plasmodium falciparum, the parasite responsible for the deadly forms of the disease, to the antimalarials conventionally used, such as chloroquine, mefloquine, sulphadoxine-pyrimethamine and amodiaquine.

Polychemoresistant P. falciparum malaria is very widespread in south-east Asia and in South America. The African continent, which bears the heaviest burden of morbidity and mortality due to malaria, is now also affected. Resistance to inexpensive monotherapies such as chloroquine and sulphadoxine-pyrimethamine is gaining ground, hence the increase in mortality. The current situation is partly due to the fact that antimalarials were used badly over the past century: antimalarials were used massively, always in monotherapy, one after the other, and generally with contempt for the regulations for the use of medicaments since they were maintained despite unacceptable resistance levels.

A new group of antimalarials—artemisinin derivatives, in particular artesunate, artemether and dihydroartemisinin—has been increasingly used over the past ten years. These compounds have a very rapid therapeutic action (decrease in parasite count and resolution of symptoms), they are effective against polychemoresistant P. falciparum malaria, they are well tolerated by patients and they reduce the carrying of gametocytes (and can therefore reduce transmission of malaria).

No resistance of the parasite to these compounds has been observed to date, although a certain decrease in sensitivity has been detected in vitro in China and in Vietnam. Used alone, the artemisinin derivatives provide recovery from P. falciparum malaria in seven days, but several studies have shown that the artemisinin derivatives combined with certain synthetic medicaments give high recovery rates in only three days with greater treatment compliance. According to some data, the use of these combinations could also substantially defer the appearance of resistance to the combined medicament in areas of low-to-moderate transmission.

In response to the increase in resistance to antimalarials, the WHO recommends, to countries which observe a resistance to conventional monotherapies, such as chloroquine, amodiaquine or sulphadoxine-pyrimethamine, the use of therapeutic combinations, preferably those which contain artemisinin derivatives, against P. falciparum malaria.

Artemisinin is a sesquiterpene lactone with two oxygen atoms bonded by a peroxide bridge above a ring with seven carbon atoms. It is mainly artemisinin derivatives which are used in therapy, for instance artemether, artesunate, arteether or dihydroartemisinin.

Artesunate corresponds to the following formula:

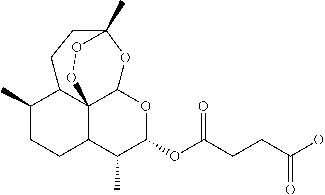

Among the antimalarials being developed is ferroquine, the structural formula of which is represented below, and which is described in application WO 96/35698

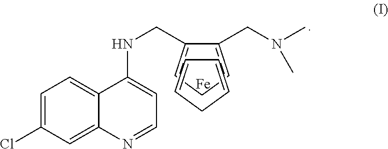

WO 2006/111647 also describes the combination of ferroquine and an artemisinin derivative, and in particular artesunate.

As a general rule, a combination of two active ingredients is produced by mixing the powders containing the active ingredients and the excipients, or else by granulating them. The most conventional method consists in producing the pharmaceutical composition corresponding to an active ingredient, and in adding the second active ingredient during the mixing or the granulation step.

However, artesunate and ferroquine are strongly incompatible if the two active ingredients are combined within one and the same tablet. In particular, the combining of ferroquine and artesunate within the same tablet inevitably causes a rapid increase in the level of artesunate degradation products within the tablet [characterized by changing the color of the artesunate, which goes from white to brown].

In the present application, the term “ferroquine” is intended to mean ferroquine in the form of a base, a salt, a hydrate or a solvate. Similarly, the term “artemisinin” is intended to mean artemisinin or a derivative thereof, for example artemether, artesunate, arteether or dihydroartemisinin, it being possible for artemisinin or a derivative thereof to be in the form of a base, a salt, a hydrate or a solvate.

A subject of the present invention is a multilayer pharmaceutical composition, characterized in that it comprises at least two layers and a minimum of two active ingredients per pharmaceutical composition, said composition being water-dispersible and comprising:
ferroquine in the form of a base, a salt, a hydrate or a solvate, as first active ingredient,
artemisinin or a derivative thereof, in the form of a base, a salt, a hydrate or a solvate, as second active ingredient,
pharmaceutically acceptable excipients.

According to one embodiment of the invention, the pharmaceutical composition is in tablet form.

The pharmaceutical composition according to the invention is a multilayer pharmaceutical composition comprising at least two layers.

In particular, this pharmaceutical composition is a two-layer pharmaceutical composition.

More particularly, this pharmaceutical composition is a three-layer pharmaceutical composition.

Thus, such a pharmaceutical composition contains therapeutically effective doses of ferroquine, or of a pharmaceutically acceptable salt, of a hydrate or of a solvate of ferroquine, and of at least one artemisinin derivative or of a derivative thereof, or of a pharmaceutically acceptable salt, of a hydrate or of a solvate of artemisinin or of a derivative thereof, and also at least one pharmaceutically acceptable excipient. Said excipients are chosen, according to the pharmaceutical form and the method of administration desired, from the usual excipients which are known to those skilled in the art.

The ferroquine can in particular be in the form of an L-tartrate salt, a dihydrochloride salt, a hydrochloride salt or a base. An example of the salt/base ratio for the L-tartrate salt of ferroquine is 1.69, and an example of the salt/base ratio for the dihydrochloride salt of ferroquine is 1.17.

The pharmaceutical composition also contains a therapeutically effective dose of artemisinin or of a derivative thereof, chosen from artemether, artesunate, arteether and dihydroartemisinin. According to one embodiment of the invention, the artemisinin derivative used is artesunate.

It is important for it to be possible for the pharmaceutical composition according to the invention to be ingested as a conventional tablet and for it not to dissolve in the oral cavity as it passes to the esophagus, even if the passage is facilitated by the simultaneous swallowing of liquid. However, malaria also affects infants and young children with immature swallowing, and it is also important for these young patients to have access to the antimalarial treatment. Thus, the pharmaceutical composition according to the invention is available in the form of tablets produced in different sizes according to the dosage and specifically suitable for the age of the patient. The size of the tablet or compression format (width×length) is expressed in millimeters (w×l mm). Since the thickness of the tablets obtained depends, firstly, on the compression format used, secondly on the amount of powder to be compressed and, finally, on the compression force applied, it is therefore variable.

The pharmaceutical composition according to the invention is also water-dispersible, making it suitable for pediatric use.

The expression “water-dispersible pharmaceutical composition” is intended to mean a pharmaceutical composition with high hardness, limited friability and rapid disintegration. The expression “water-dispersible” does not limit the scope of the invention to a dispersion of the pharmaceutical composition in water, but should be understood to be the dispersion of the pharmaceutical composition in an aqueous liquid vehicle, such as, for example, water, fruit juice, milk or alternatively a fizzy drink.

In particular, all the tests carried out on the 10 dosages targeted a disintegration time of at most 120±15 seconds in purified water at ambient temperature.

Moreover, the hardness of the present composition is dependent on the size of the tablet (compression format). For tablets having a compression format of 5.50×9.60 mm, the hardness of the present composition is between 50 and 160 N, preferably between 60 and 150 N, and even more preferably between 70 and 140 N, for example 93.8 N.

For tablets having a compression format of 6.95×12.17 mm, the hardness of the present composition is between 100 and 175N, preferably between 110 and 165 N, and even more preferably between 115 and 160N, for example 132 N.

For tablets having a compression format of 8.76×15.3 mm, the hardness of the present composition is between 110 and 230 N, preferably between 120 and 220 N, and even more preferably between 130 and 210 N, for example 169 N.

For tablets having a compression format of 10×17.5 mm, the hardness of the present composition is between 140 and 260 N, preferably between 160 and 240 N, and even more preferably between 170 and 230 N, for example 198 N.

The hardness is measured according to the method described below: the tablet is placed against the fixed jaw of a durometer. The other, mobile, jaw moves by means of a motorized drive system and applies, to the tablet, a pressure which constantly increases. The increase in pressure is monitored electronically by the durometer and continues to be applied to the sample until an equal or greater value is measured. At the time of break, the resistance of the tablet to the pressure applied decreases. The measuring system detects this decrease and the highest resistance value is then displayed and is validated as being the breaking strength value for the tablet.

The friability of the pharmaceutical composition is also dependent on the size of the tablet (compression format) and on the compression strength applied, having an effect on the disintegration time of the tablets obtained.

For tablets having a compression format of 5.50×9.60 mm, the friability of the pharmaceutical composition after 4 minutes is, according to one of the aspects of the invention, between 0 and 0.3%, preferably between 0 and 0.2%, even more preferably between 0 and 0.15%, for example 0.11%.

For tablets having a compression format of 6.95×12.17 mm, the friability of the pharmaceutical composition after 4 minutes is, according to one of the aspects of the invention, between 0 and 0.3%, and even more preferably between 0 and 0.2%, for example 0.11%.

For tablets having a compression format of 8.76×15.3 mm, the friability of the pharmaceutical composition after 4 minutes is, according to one of the aspects of the invention, between 0 and 0.3%, and even more preferably between 0 and 0.2%, for example 0.17%.

For tablets having a compression format of 10×17.5 mm, the friability of the pharmaceutical composition after 4 minutes is, according to one of the aspects of the invention, between 0 and 0.5%, preferably between 0 and 0.4%, even more preferably between 0 and 0.3%, for example 0.21%.

The friability of the tablets is measured after 100 rotations according to the method described in the European Pharmacopoeia, 6^{th} Edition, 2010, Chapter 2.9.7.

The disintegration is measured according to the method described in the European Pharmacopoeia, 6^{th} Edition, 2010, Chapter 2.9.1. The tablets obtained, irrespective of the formats and the dosages, comply with the standard applied with regard to “dispersible tablets”.

The fineness of dispersion is also, in one embodiment, consistent with the following definition: two tablets are placed in 100 ml of purified water, which is stirred until complete dispersion. The dispersion obtained is homogeneous and passes through a screen with a nominal mesh size of 710 μm. The fineness of dispersion is measured according to the method described in the European Pharmacopoeia, 6^{th} Edition, 2010, Chapter 7, relating to the definition of dispersible tablets.

According to one embodiment, the tablet according to the invention comprises two layers, one comprising ferroquine in the form of a base, a salt, a hydrate or a solvate, and the other comprising artesunate in the form of a base, a salt, a hydrate or a solvate.

According to another embodiment, the tablet according to the invention comprises three layers, one comprising ferroquine in the form of a base, a salt, a hydrate or a solvate, and a second comprising artesunate in the form of a base, a salt, a hydrate or a solvate, these two layers being separated by an isolating layer containing no active ingredient (neutral).

The layers may be identical or different in terms of weight or of volume, and they may or may not all be visible from the exterior. For example, it is possible to have an upper half of a tablet according to the invention comprising ferroquine, and a lower half comprising artemisinin or a derivative thereof, the two halves being the same color and having the same appearance, and therefore indistinguishable by the patient. The two halves may alternately be of different color and/or have a different appearance.

In the case of a tablet comprising three layers, the two layers containing the 2 active ingredients will be separated by an isolating layer which is the same color and has the same appearance as one of the two layers comprising one of the active ingredients, said layers therefore remaining indistinguishable by the patient. The neutral isolating layer makes it possible to separate the two layers containing the incompatible active ingredients. It therefore limits the degradation of the active ingredients within the tablet and therefore makes it possible to obtain greater stability of the formulation. The two halves and also the intermediate isolating layer may alternatively be of different color and/or have a different appearance.

It is also envisaged that the tablet be a “sandwich” of layers comprising different active ingredients, for example a layer of artemisinin or of a derivative thereof, between two layers of ferroquine, or vice versa.

Another aspect of the invention is that one of the layers containing a first active ingredient is completely coated with the other layer comprising the second active ingredient.

According to another subject, the invention consists of a water-dispersible multilayer pharmaceutical composition comprising at most two active ingredients per pharmaceutical composition, said composition comprising:
ferroquine as first active ingredient,
artemisinin or a derivative thereof as second active ingredient, and
pharmaceutically acceptable excipients,
   the level of disintegrating agent being less than 5% by weight of the tablet, expressed relative to the total mass of the tablet.

According to one embodiment of the invention, the level of disintegrating agents of the pharmaceutical composition, in particular of the tablet, is less than 5%, and in particular less than 3.5%, relative to the total mass of the tablet.

According to another embodiment of the invention, the level of disintegrating agent in each of the layers is less than 2.5%, relative to the total mass of the tablet, for example less than 2%.

The pharmaceutical composition according to the invention has:
a disintegration time of 120±15 seconds;
a friability of less than 0.5%;
a level of disintegrating agent of less than 5% relative to the total mass of the tablet;
a level of disintegrating agent in each of the layers of less than 2.5% relative to the mass;
a hardness of between:
50 and 160 N when the pharmaceutical composition has a compression format of 5.50×9.60 mm;
100 and 175 N when the pharmaceutical composition has a compression format of 6.95×12.17 mm;
110 and 230 N when the pharmaceutical composition has a compression format of 8.76×15.30 mm;
140 and 260 N when the pharmaceutical composition has a compression format of 10×17.50 mm.

More particularly, the pharmaceutical composition according to the invention has:
a disintegration time of 120±15 seconds;
a friability of less than 0.4%;
a level of disintegrating agent of less than 3.5% relative to the total mass of the tablet;
a level of disintegrating agent in each of the layers of less than 2% relative to the mass;
a hardness of between:
70 and 140 N when the pharmaceutical composition has a compression format of 5.50×9.60 mm;
115 and 160 N when the pharmaceutical composition has a compression format of 6.95×12.17 mm;
130 and 210 N when the pharmaceutical composition has a compression format of 8.76×15.30 mm;
170 and 230 N when the pharmaceutical composition has a compression format of 10×17.50 mm.

The applicant has found, surprisingly, that the pharmaceutical compositions according to the invention, despite high tablet hardnesses, very low friabilities, and a level of disintegrating agent of less than 5%, or even less than 3.5%, relative to the total mass of the tablet, are nevertheless hydrodispersible. The term “hydrodispersible” or “water-dispersible” is intended to mean the definition in the European Pharmacopoeia, which stipulates that dispersible tablets are uncoated tablets or film-coated tablets intended to be dispersed in water before administration, giving a homogeneous dispersion.

According to another embodiment of the invention, the pharmaceutical composition comprises:
compounds of the internal phase for the ferroquine layer:
   between 5% and 30% of ferroquine,
   between 0.2% and 1.0% of disintegrating agent,
   between 10% and 40% of diluent,
optionally, compounds of the external phase for the ferroquine layer:
   between 0.1% and 1% of lubricant,
   between 0 and 4.0% of sweetener,
compounds of the internal phase for the layer of artemisinin or a derivative thereof:
   between 5% and 20% of artemisinin or a derivative thereof,
   between 0 and 10% of diluent,
   between 8% and 16% of codiluent,
   between 0.5% and 2% of binder,
   between 0 and 2% of disintegrating agent,
optionally, compounds of the external phase for the layer of artemisinin or a derivative thereof:
   between 0 and 1% of lubricant,
   between 0 and 0.3% of flow agent,
compounds for the neutral isolating layer:
   between 10% and 30% of diluent,
   between 0 and 10% of codiluent,
   between 0 and 0.5% of lubricant,
   between 0 and 2% of disintegrating agent,
      it being understood that, if the pharmaceutical composition does not comprise an external phase, it should contain from 0.5% to 2% of lubricant.

The pharmaceutically acceptable excipients comprise a disintegrating agent which can be chosen from sodium carboxymethyl starch, sodium croscarmellose, calcium carboxymethylcellulose or sodium carboxymethylcellulose, pregelatinized starch, crospovidone or alginic acid or one of its derivatives.

Examples of binders which can be used in the invention are polyethylene glycol, pregelatinized starch, copovidone, maltodextrins, hydroxypropylcellulose, guar gum, alginates, for example sodium alginates, povidone (polyvinyl-pyrrolidone PVP K30), carbomers, methylcellulose, dextrins, hydroxyethylmethylcellulose, hydroxypropylcellulose, hypromellose, ethylcellulose, polydextrose, gelatin, propylene glycol or else polymethacrylates.

The diluent can be chosen from microcrystalline cellulose PH 112, starch (maize, rice, potato, in particular), pregelatinized starch, maltose, mannitol, maltitol, xylitol, lactitol, sorbitol, fructose, lactose (whether it is anhydrous, monohydrate or atomized), dextrin or a derivative thereof, calcium carbonate, calcium lactate, calcium phosphate, calcium sulfate or sucrose, for example.

Examples of lubricants which can be used in the context of the invention are magnesium stearate, hydrogenated castor oil, glyceryl palmitostearate, polyoxyethylene stearates, glyceryl behenate, sodium lauryl sulfate, calcium stearate, leucine, sodium stearyl fumarate, poloxamer, glyceryl monostearate or else polyethylene glycol.

Sweetener can be chosen from potassium acesulfame, sucrose, sucralose, aspartame, neohesperidin dihydrochalcone, thaumatin, neotame, tagatose, sodium saccharin, sodium cyclamate, maltose, mannitol, maltitol, xylitol, lactitol, sorbitol, fructose, lactose (whether it is anhydrous, monohydrate or atomized) or else trehalose.

The flow agent can be chosen from colloidal silica, talc, magnesium silicate, calcium stearate or calcium phosphate, for example.

According to one embodiment, the pharmaceutical composition comprises:
in the compounds of the internal phase for the ferroquine layer, sodium carboxymethyl starch as disintegrating agent, microcrystalline cellulose as diluent;
in the compounds of the external phase for the ferroquine layer, magnesium stearate as lubricant, potassium acesulfame as sweetener;
in the compounds of the internal phase for the artesunate layer, calcium carbonate as diluent, microcrystalline cellulose as codiluent, sodium croscarmellose as disintegrating agent, povidone as binder;
in the compounds of the external phase for the artesunate layer, colloidal silica as flow agent, magnesium stearate as lubricant;
in the compounds of the neutral isolating layer, calcium carbonate as diluent, microcrystalline cellulose as codiluent, magnesium stearate as lubricant and sodium carboxymethyl starch as disintegrating agent.

The daily doses of each of the two active ingredients of the combination according to the invention can be the following:
ferroquine: between 1 and 10 mg/kg/day, for example 2, 4 or 6 mg/kg/day;
artemisinin derivative: between 1 and 10 mg/kg/day, for example between 2 and 6 mg/kg/day, or else approximately 4 mg/kg/day.

There may be specific cases where higher or lower dosages are appropriate; such dosages do not depart from the context of the invention. According to customary practice, the appropriate dosage for each patient is determined by the physician according to the method of administration and the weight and response of said patient.

The pharmaceutical composition according to the invention is intended to be administered for 3 consecutive days, in one or more daily intakes of each of the two active ingredients, preferably a single intake per day. This treatment time limited to 3 days is particularly advantageous, in comparison with the 7 days recommended for a monotherapy with the artemisinin derivatives, in that it enables better treatment compliance by the patients, thus avoiding premature interruptions of the treatment which induce, in the long term, resistance of the parasite.

By way of examples of a pharmaceutical composition according to the invention, the following tablets can be envisaged:

TABLE 1
Example 1:

| | |
|---|---|
| Amount | |
| (mg/tab) | Function |

Ferroquine layer (internal phase)

| | | |
|---|---|---|
| Ferroquine | 200.00 | Active ingredient |
| Sodium carboxymethyl starch | 7.50 | Disintegrating agent |
| Microcrystalline cellulose | 273.25 | Diluent |

Ferroquine layer (external phase)

| | | |
|---|---|---|
| Magnesium stearate | 4.25 | Lubricant |
| Potassium acesulfame | 15.00 | Sweetener |

Neutral isolating layer

| | | |
|---|---|---|
| Calcium carbonate | 152 | Diluent |
| Microcrystalline cellulose | 38 | Codiluent |
| Magnesium stearate | 2 | Lubricant |
| Sodium carboxymethyl starch | 8 | Disintegrating agent |

Artesunate layer (internal phase)

| | | |
|---|---|---|
| Artesunate | 100.00 | Active ingredient |
| Calcium carbonate | 54.00 | Diluent |
| Microcrystalline cellulose | 90.00 | Codiluent |
| Sodium croscarmellose | 9.80 | Disintegrating agent |
| Povidone | 8.00 | Binder |

Artesunate layer (external phase)

| | | |
|---|---|---|
| Colloidal silica | 0.75 | Flow agent |
| Magnesium stearate | 3.00 | Lubricant |

Such a tablet is, for example, compressed while adhering to the following parameters:
compacting pressure for the internal phase of the ferroquine layer: 45 bar;
compression format: 17.5×10 mm;
compression force applied to the first layer (artesunate layer): 0 kN;
compression force applied to the second layer (neutral layer): 2 kN;
final compression force for the second layer (ferroquine layer): 24 kN.

TABLE 2

| | Amount (mg/tab) | | |
|---|---|---|---|
| Examples | 2 | 3 | 4 |

Ferroquine layer (internal phase)

| | | | |
|---|---|---|---|
| Ferroquine | 150 | 100 | 50 |
| Sodium carboxymethyl starch | 5.63 | 3.75 | 1.88 |
| Microcrystalline cellulose | 204.94 | 136.63 | 68.31 |

Ferroquine layer (external phase)

| | | | |
|---|---|---|---|
| Magnesium stearate | 3.19 | 2.13 | 1.06 |
| Potassium acesulfame | 11.25 | 7.50 | 3.75 |

Neutral isolating layer

| | | | |
|---|---|---|---|
| Calcium carbonate | 152 | 152 | 152 |
| Microcrystalline cellulose | 38 | 38 | 38 |
| Magnesium stearate | 2 | 2 | 2 |
| Sodium carboxymethyl starch | 8 | 8 | 8 |

Artesunate layer (internal phase)

| | | | |
|---|---|---|---|
| Artesunate | 100 | 100 | 100 |
| Calcium carbonate | 54 | 54 | 54 |
| Microcrystalline cellulose | 90 | 90 | 90 |
| Sodium croscarmellose | 9.80 | 9.80 | 9.80 |
| Povidone | 8 | 8 | 8 |

Artesunate layer (external phase)

| | | | |
|---|---|---|---|
| Colloidal silica | 0.75 | 0.75 | 0.75 |
| Magnesium stearate | 3 | 3 | 3 |

Such a tablet is, for example, compressed while adhering to the following parameters:
compacting pressure for the internal phase of the ferroquine layer: 45 bar;
compression format: 8.76×15.30 mm;
compression force applied to the first layer (artesunate layer): 0.2 kN;
compression force applied to the second layer (neutral layer): 0.4 kN;
final compression force for the third layer (ferroquine layer): 17.5 kN.

TABLE 3

| | Amount (mg/tab) | | |
|---|---|---|---|
| Examples | 5 | 6 | 7 |

Ferroquine layer (internal phase)

| | | | |
|---|---|---|---|
| Ferroquine | 75 | 50 | 25 |
| Sodium carboxymethyl starch | 2.81 | 1.88 | 0.94 |
| Microcrystalline cellulose | 102.47 | 68.31 | 34.16 |

Ferroquine layer (external phase)

| | | | |
|---|---|---|---|
| Magnesium stearate | 1.59 | 1.06 | 0.53 |
| Potassium acesulfame | 5.63 | 3.75 | 1.88 |

Neutral isolating layer

| | | | |
|---|---|---|---|
| Calcium carbonate | 76 | 76 | 76 |
| Microcrystalline cellulose | 19 | 19 | 19 |
| Magnesium stearate | 1 | 1 | 1 |
| Sodium carboxymethyl starch | 4 | 4 | 4 |

Artesunate layer (internal phase)

| | | | |
|---|---|---|---|
| Artesunate | 50 | 50 | 50 |
| Calcium carbonate | 27 | 27 | 27 |
| Microcrystalline cellulose | 45 | 45 | 45 |
| Sodium croscarmellose | 4.9 | 4.9 | 4.9 |
| Povidone | 4 | 4 | 4 |

Artesunate layer (external phase)

| | | | |
|---|---|---|---|
| Colloidal silica | 0.38 | 0.38 | 0.38 |
| Magnesium stearate | 1.50 | 1.50 | 1.50 |

Such a tablet is, for example, compressed while adhering to the following parameters:
compacting pressure for the internal phase of the ferroquine layer: 45 bar;
compression format: 6.95×12.17 mm;
compression force applied to the first layer (artesunate layer): 0.2 kN;
compression force applied to the second layer (neutral layer): 0.2 kN;
final compression force for the third layer (ferroquine layer): 13.4 kN.

TABLE 4

| | Amount (mg/tab) | | |
|---|---|---|---|
| Examples | 8 | 9 | 10 |

Ferroquine layer (internal phase)

| | | | |
|---|---|---|---|
| Ferroquine | 37.5 | 25 | 12.5 |
| Sodium carboxymethyl starch | 1.41 | 0.94 | 0.47 |
| Microcrystalline cellulose | 51.23 | 34.16 | 17.08 |

Ferroquine layer (external phase)

| | | | |
|---|---|---|---|
| Magnesium stearate | 0.80 | 0.53 | 0.27 |
| Potassium acesulfame | 2.81 | 1.88 | 0.94 |

Neutral isolating layer

| | | | |
|---|---|---|---|
| Calcium carbonate | 38 | 38 | 38 |
| Microcrystalline cellulose | 9.5 | 9.5 | 9.5 |
| Magnesium stearate | 0.5 | 0.5 | 0.5 |
| Sodium carboxymethyl starch | 2 | 2 | 2 |

Artesunate layer (internal phase)

| | | | |
|---|---|---|---|
| Artesunate | 25 | 25 | 25 |
| Calcium carbonate | 13.50 | 13.50 | 13.50 |
| Microcrystalline cellulose | 22.50 | 22.50 | 22.50 |
| Sodium croscarmellose | 2.45 | 2.45 | 2.45 |
| Povidone | 2 | 2 | 2 |

Artesunate layer (external phase)

| | | | |
|---|---|---|---|
| Colloidal silica | 0.19 | 0.19 | 0.19 |
| Magnesium stearate | 0.75 | 0.75 | 0.75 |

Such a tablet is, for example, compressed while adhering to the following parameters:
compacting pressure for the internal phase of the ferroquine layer: 45 bar;

compression format: 5.5×9.6 mm;

compression force applied to the first layer (artesunate layer): 0.2 kN;

compression force applied to the second layer (neutral layer): 0.2 kN;

final compression force for the third layer (ferroquine layer): 8.75 kN.

According to another subject, the invention consists of a method for producing the pharmaceutical composition.

The grains for compression of the ferroquine layer are prepared separately from the grains for compression of the layer of artemisinin or derivatives thereof and from the grains of the neutral layer.

The grain for compression of the ferroquine layer is prepared by
premixing the internal phase compounds,
compacting the internal phase, and
mixing the compacted grain with the external phase excipients.

The grain for compression of the layer of artemisinin or derivatives thereof is prepared by mixing the active ingredient and the excipients of the internal and external phase. The grain of the neutral layer is, for its part, prepared by simple mixing of all its constituents.

The compression step is then carried out on a multilayer tableting machine, for example of the Fette P102i or Hata HT45 type.

More particularly, the grain for compression of the ferroquine layer is prepared according to the following steps:
weighing out of the internal phase compounds,
screening of these compounds,
premixing of these compounds in order to obtain a homogeneous distribution of the active ingredient and of the excipients,
compacting of the internal phase using a roller compactor,
weighing out of the external phase excipients,
mixing of the internal phase compacted grain and of the external phase excipients so as to obtain a grain ready for compression,
   and the grain for compression of the layer of artemisinin or derivatives thereof is prepared according to the following procedure:
weighing out of the internal phase compounds,
premixing of the internal phase compounds,
weighing out of the magnesium stearate and the colloidal silica and then screening thereof,
final mixing of the magnesium stearate and colloidal silica compounds so as to obtain a mixture ready for compression.

The grain for compression of the neutral layer is prepared according to the following procedure:
weighing out of all the constituents,
screening of these constituents,
mixing of all the constituents.

The method for producing the multilayer tablets, and in particular three-layer, ferroquine-neutral-artemisinin or a derivative thereof, tablets can thus be exemplified as described below:

a) preparation of the ferroquine layer:
a1) weighing out of the ferroquine, a diluent and a disintegrating agent,
a2) premixing for 15 minutes at 7 rpm,
a3) optionally, calibrating on a rotary calibrating device with a 1.5 mm screen,
a4) mixing for 30 minutes at 7 rpm,
a5) compacting on a roller compactor, for example an Alexanderwerk WP 50 roller compactor with screens of 2.5 and 1.25 mm,
a6) optionally, mixing of a lubricant and secondarily of a sweetener, then calibration of this ingredient or these two ingredients on a rotary calibration of device with a 1 mm screen,
a7) if step a6) has been carried out, mixing of the compounds resulting from step a6) and of the compounds resulting from step a5),
a8) optionally, mixing in a Robotainer mixer, for 30 minutes at 7 rpm;
b) preparation of the layer of artemisinin or a derivative thereof:
b1) weighing out of the active ingredient (artemisinin or a derivative thereof), a diluent, a disintegrating agent, a binder and a codiluent,
b2) optionally, mixing in an inverting mixer, or a CMA rotary mixer, for 15 minutes at 7 rpm,
b3) optionally, calibrating on a CMA rotary calibrating device with a screen of, for example, 1 mm,
b4) mixing of a lubricant and of a flow agent, then optionally calibration of these two ingredients with a 1.0 mm screen,
b5) optionally, mixing in a Robotainer mixer for 15 minutes at 7 rpm,
b6) mixing in a Robotainer mixer for 30 minutes at 7 rpm,
c) preparation of the neutral layer:
c1) weighing out of the two diluents, the disintegrating agent and the lubricant,
c2) optionally, calibrating on a rotary calibrating device with a 1.0 mm screen,
c3) optionally, mixing in an inverting mixer, or a CMA rotary mixer, for 30 minutes at 7 rpm,
d) compression of the mixtures obtained:
in a9) if this step has been carried out, or, where appropriate, in the final step carried out in a), and
in b6), and
in c3),
   for example in a multilayer tableting machine, for example of the Fette P102i or Hata HT45 type.

According to one embodiment of the invention, the agents mentioned in the steps hereinafter are, respectively:
step a1): microcrystalline cellulose PH112, sodium carboxymethyl starch,
step a6) or, where appropriate, in the last step carried out in a): magnesium stearate and potassium acesulfame,
step b1): calcium carbonate, sodium croscarmellose, povidone (PVP K30) and microcrystalline cellulose PH112,
step b4): magnesium stearate and anhydrous colloidal silica,
step c1): microcrystalline cellulose, calcium carbonate, sodium carboxymethyl starch, magnesium stearate.

By way of example, a pharmaceutical composition according to the invention which has the composition mentioned in the table and which is produced according to the above-mentioned method has the following characteristics:
compression format: 17.5×10 mm,
average tablet hardness (n=10): 198N,
friability after 4 minutes (%) according to the European Pharmacopoeia (n=10): 0.21%
disintegration time according to the European Pharmacopoeia (n=6) in purified water at ambient temperature: 123 seconds,
conformity with respect to fineness of dispersion according to the European Pharmacopoeia: conforms.

What is claimed is:

1. A multilayer tablet comprising at least two layers and a minimum of two active ingredients per tablet, said tablet being water-dispersible and comprising:
a layer comprising ferroquine in the form of a base, a salt, a hydrate or a solvate,
another layer comprising artesunate in the form of a base, a salt, a hydrate or a solvate, and
pharmaceutically acceptable excipients.

2. A tablet according to claim 1, wherein the tablet is a two-layer tablet.

3. A tablet according to claim 1, wherein the tablet is a three-layer tablet.

4. A tablet according to claim 3, wherein the two layers comprising active ingredients are separated by a neutral isolating layer containing no active ingredient.

5. A tablet according to claim 1, wherein the tablet has a disintegration time of at most 120±15 seconds.

6. A tablet according to claim 1, wherein the tablet has a compression format of 5.50×9.60 mm and a hardness of between 50 and 160 N.

7. A tablet according to claim 6, wherein the tablet has a compression format of 5.50×9.60 mm and a hardness of between 60 and 150 N.

8. A tablet according to claim 7, wherein the tablet has a compression format of 5.50×9.60 mm and a hardness of between 70 and 140 N.

9. A tablet according to claim 1, wherein the tablet has a compression format of 6.95×12.17 mm and a hardness of between 100 and 175 N.

10. A tablet according to claim 9, wherein the tablet has a compression format of 6.95×12.17 mm and a hardness of between 110 and 165 N.

11. A tablet according to claim 10, wherein the tablet has a compression format of 6.95×12.17 mm and a hardness of between 115 and 160 N.

12. A tablet according to claim 1, wherein the tablet has a compression format of 8.76×15.3 mm and a hardness of between 110 and 230 N.

13. A tablet according to claim 12, wherein the tablet has a compression format of 8.76×15.3 mm and a hardness of between 120 and 220 N.

14. A tablet according to claim 13, wherein the tablet has a compression format of 8.76×15.3 mm and a hardness of between 130 and 210 N.

15. A tablet according to claim 1, wherein the tablet has a compression format of 10×17.5 mm and a hardness of between 140 and 260 N.

16. A tablet according to claim 15, wherein the tablet has a compression format of 10×17.5 mm and a hardness of between 160 and 240 N.

17. A tablet according to claim 16, wherein the tablet has a compression format of 10×17.5 mm and a hardness of between 170 and 230 N.

18. A tablet according to claim 1, wherein the tablet has a compression format of 5.50×9.60 mm and a friability after 4 minutes of between 0 and 0.30%.

19. A tablet according to claim 18, wherein the tablet has a compression format of 5.50×9.60 mm and a friability of between 0 and 0.20%.

20. A tablet according to claim 19, wherein the tablet has a compression format of 5.50×9.60 mm and a friability of between 0 and 0.15%.

21. A tablet according to claim 1, wherein the tablet has a compression format of 6.95×12.17 mm and a friability after 4 minutes of between 0 and 0.30%.

22. A tablet according to claim 21, wherein the tablet has a compression format of 6.95×12.17 mm and a friability of between 0 and 0.20%.

23. A tablet according to claim 1, wherein the tablet has a compression format of 8.76×15.3 mm and a friability after 4 minutes of between 0 and 0.30%.

24. A tablet according to claim 23, wherein the tablet has a compression format of 8.76×15.3 mm and a friability of between 0 and 0.20%.

25. A tablet according to claim 1, wherein the tablet has a compression format of 10×17.5 mm and a friability after 4 minutes of between 0 and 0.5%.

26. A tablet according to claim 25, wherein the tablet has a compression format of 10×17.5 mm and a friability of between 0 and 0.4%.

27. A tablet according to claim 26, wherein the tablet has a compression format of 10×17.5 mm and a friability of between 0 and 0.3%.

28. A tablet according to claim 1, wherein the tablet comprises a level of disintegrating agent of less than 5% by mass, relative to the total mass of the tablet.

29. A tablet according to claim 28, wherein the tablet comprises a level of disintegrating agent of less than 3.5% by mass, relative to the total mass of the tablet.

30. A tablet according to claim 28, wherein the tablet comprises a level of disintegrating agent of less than 2% by mass, relative to the total mass of the tablet in each of the layers.

31. A tablet according to claim 1, wherein the tablet has:
a disintegration time of 120±15 seconds;
a friability of less than 0.5%; a level of disintegrating agent of less than 5% by mass relative to the total mass of the tablet;
a level of disintegrating agent in each of the layers of less than 2.5% by mass relative to the total mass of the tablet; and
a hardness of between:
50 and 160 N when the tablet has a compression format of 5.50×9.60 mm
or
100 and 175 N when the tablet has a compression format of 6.95×12.17 mm
or
110 and 230 N when the tablet has a compression format of 8.76×15.30 mm
or
140 and 260 N when the tablet has a compression format of 10×17.50 mm.

32. A tablet according to claim 31, wherein the tablet has:
a disintegration time of 120±15 seconds;
a friability of less than 0.4%;
a level of disintegrating agent of less than 3.5% by mass relative to the total mass of the tablet;
a level of disintegrating agent in each of the layers of less than 2% by mass relative to the total mass of the tablet; and
a hardness of between:
70 and 140 N when the tablet has a compression format of 5.50×9.60 mm
or
115 and 160 N when the tablet has a compression format of 6.95×12.17 mm
or
130 and 210 N when the tablet has a compression format of 8.76×15.30 mm
or
170 and 230 N when the tablet has a compression format of 10×17.50 mm.

33. A tablet according to claim 1, wherein:
the layer comprising ferroquine comprises an internal phase containing:
between 5% and 30% of ferroquine,
between 0.2% and 1.0% of disintegrating agent,
between 10% and 40% of diluent,
and, optionally, an external phase containing:
between 0.1% and 1% of lubricant,
between 0 and 4.0% of sweetener;
the layer comprising artemisinin or a derivative thereof comprises an internal phase containing:
between 5% and 20% of artemisinin or a derivative thereof,
between 0 and 10% of diluent,
between 8% and 16% of codiluent,
between 0.5% and 2% of binder,
between 0 and 2% of disintegrating agent,
and, optionally, an external phase containing:
between 0 and 1% of lubricant,
between 0 and 0.3% of flow agent; and
a neutral isolating layer comprising:
between 10% and 30% of diluent,
between 0 and 10% of codiluent,
between 0 and 0.5% of lubricant,
between 0 and 2% of disintegrating agent,
it being understood that, if the tablet does not comprise an external phase, it should contain from 0.5% to 2% of lubricant, all percentages by mass based on the total mass of the tablet.

34. A tablet according to claim 33, wherein it comprises:
in the internal phase for the ferroquine layer, sodium carboxymethyl starch as disintegrating agent, and microcrystalline cellulose as diluent,
in the external phase for the ferroquine layer, magnesium stearate as lubricant, and potassium acesulfame as sweetener,
in the internal phase for the artesunate layer, calcium carbonate as diluent, microcrystalline cellulose as codiluent, sodium croscarmellose as disintegrating agent of the layer of artemisinin or a derivative thereof, and povidone as binder of the layer of artemisinin or a derivative thereof,
in the external phase for the artesunate layer, colloidal silica as flow agent, and magnesium stearate as lubricant, and
in the neutral isolating layer, calcium carbonate as diluent, microcrystalline cellulose as codiluent, magnesium stearate as lubricant and sodium carboxymethyl starch as disintegrating agent.

35. A method for producing a tablet according to claim 1, comprising the following steps:
a) preparation of the ferroquine layer:
a1) weighing out of the ferroquine, a diluent and a disintegrating agent,
a2) premixing for 15 minutes at 7 rpm,
a3) optionally, calibrating on a rotary calibrating device with a 1.5 mm screen,
a4) mixing for 30 minutes at 7 rpm,
a5) compacting on a roller compactor,
a6) optionally, mixing of a lubricant and secondarily of a sweetener, then calibration of this ingredient or these two ingredients on a rotary calibrating device with a 1 mm screen,
a7) if step a6) has been carried out, mixing of the compounds resulting from step a6) and of the compounds resulting from step a5), and
a8) optionally, mixing in a Robotainer® mixer for 30 minutes at 7 rpm;
b) preparation of the layer of artemisinin or a derivative thereof:
b1) weighing out of the active ingredient (artemisinin or a derivative thereof), a diluent, a disintegrating agent, a binder and a codiluent,
b2) optionally, mixing in an inverting mixer, or a CMA rotary mixer, for 15 minutes at 7 rpm,
b3) optionally, calibrating on a CMA rotary calibrating device,
b4) mixing of a lubricant and of a flow agent, then optionally calibrating of these two ingredients with a 1.0 mm screen,
b5) optionally, mixing in a Robotainer® mixer for 15 minutes at 7 rpm,
b6) mixing in a Robotainer® mixer for 30 minutes at 7 rpm;
c) preparation of the neutral layer:
c1) weighing out of the two diluents, the disintegrating agent and the lubricant,
c2) optionally, calibrating on a rotary calibrating device with a 1.0 mm screen,
c3) optionally, mixing in an inverting mixer, or a CMA rotary mixer, for 30 minutes at 7 rpm; and
d) compression of the mixtures obtained in a9) if this step has been carried out, or, where appropriate, in the final step carried out in a),
and
in b6), and
in c3).
